(19) United States
(12) Patent Application Publication
(10) Pub. No.: US 2018/0228664 A1
(43) Pub. Date: Aug. 16, 2018

(54) LINE-UP OF ABSORBENT ARTICLES HAVING MATCHING WAIST CIRCUMFERENCES

(71) Applicant: The Procter & Gamble Company, Cincinnati, OH (US)

(72) Inventors: Ting Hou, Beijing (CN); Thorsten Rinnert, Schwalbach (DE); Andrew James Sauer, Cincinnati, OH (US)

(21) Appl. No.: 15/897,653

(22) Filed: Feb. 15, 2018

(30) Foreign Application Priority Data
Feb. 15, 2017 (WO) CN2017/073601

(51) Int. Cl. A61F 13/49; A61F 13/56; A61F 13/78

(52) U.S. Cl. CPC A61F 13/49007; A61F 2013/530576; A61F 13/78; A61F 13/5622

(57) ABSTRACT

A line-up of at least three hygiene absorbent articles of different sizes, each article comprising a pair of fasteners separated by a wingspan (W) that can be fastened to a fastening area (44) on the front region of the article so that the front region and the waist region form a waist opening. The fastening area in the front region comprises an innermost pair of fastening indicia (A-A), an outermost pair of fastening indicia (C-C) and preferably at least one intermediate pair of fastening indicia (B-B). The outer waist circumference of the article of a given size matches within 30 mm the inner waist circumference of the article of the next upper size in the line-up, the circumferences being calculated by adding the wingspan and the spacing between the innermost pair or outermost pair of indicia respectively.

### CROSS REFERENCE TO RELATED APPLICATION

This application claims the benefit of PCT filing CN2017/073601, filed Feb. 15, 2017, the substance of which is incorporated by reference.

### FIELD OF THE INVENTION

The invention relates to a line-up of personal hygiene absorbent articles of different sizes, in particular a line-up of baby diapers having different sizes. The invention is directed to an improved placement of fastening indicia across the sizes to provide for a better fastening of the articles as the wearer moves from one size to the next in the line-up.

### BACKGROUND OF THE INVENTION

Absorbent articles for personal hygiene are designed to absorb and contain body exudates, in particular large quantity of urine. So-called taped or refastenable diapers, typically comprise a pair of fasteners respectively disposed on each side of the back region of the diaper that can be releasably fastened to a fastening area on the front region of the diaper. A caregiver will typically open the diaper flat on a horizontal surface such as a changing mat or a bed with the internal surface of the diaper facing upwards. The buttocks of the baby (or of the adult for adult incontinence product) are placed towards the middle of the diaper, with the back of the wearer lying on the back region of the diaper. The front region of the diaper is then folded over the front waist of the wearer, and the diaper is then closed around the wearer by folding the remaining right side and left side of the back region on the front region of the diaper and fastening the fasteners to the fastening area on the front region of the diaper. The diaper is maintained in a closed position by attaching the fasteners to the fastening area on the front region of the diaper.

The fastening system used in diapers is typically of the hook-and-loop type with the fastening tapes comprising the hooks being releasably fastened to a fastening area on the front waist of the diaper comprising the loops. The tapes may be attached directly to the chassis of the diapers on each side of the back waist edge of the diaper or indirectly via back ears which are intermediate pieces of material, as is known in the art. The loops may be provided on a dedicated piece of material attached to the front waist of the diaper, herein called landing zone, or if a nonwoven is used as external surface for the backsheet cover, the fibers of the nonwoven may provide enough anchoring possibilities for the hooks of the fasteners. Some exemplary fastening systems are disclosed in U.S. Pat. No. 3,848,594, U.S. Pat. No. 4,662,875, U.S. Pat. No. 4,846,815, U.S. Pat. No. 4,894,060, U.S. Pat. No. 4,946,527, U.S. Pat. No. 5,151,092 and U.S. Pat. No. 5,221,274 (Buell et al.). An exemplary interlocking fastening system is also disclosed in US 6,432,098. The fastening system may also provide a means for holding the article in a disposal configuration as disclosed in U.S. Pat. No. 4,963,140 (Robertson et al.).

The fastening area is typically disposed adjacent the front edge of the absorbent article and covers a region extending along a portion of the circumference of the front region of the diaper. The tape fasteners can be fastened more or less tightly around the wearer's waist by varying their position on the fastening area. Fasteners are typically attached to elastic components such as elastic back ears or an elastic waist feature along the back edge of the article. Attaching the fasteners at different positions on the fastening area typically results in different amount of tension along the waist opening of the article. Young parents in particular may have difficulties determining the optimum placement of the fasteners to provide sufficient tension or tightness on the waist opening to minimize fluid leakage of the diaper while preventing pressure marks on the skin of the baby.

Most manufacturers place fastening indicia on the fastening area of the diaper. The fastening indicia are visible cue or guide that provide an indication to the caregivers of possible placements for the fastening tapes. The fastening indicia may typically comprise several pairs of individual indicia disposed symmetrically relative to the longitudinal axis of the diaper and extending along the circumferences of the front region. For example, each indicia may comprise a regular geometrical shape such as a circle, with a clearly defined center. A numerical indicia may be typically present in the circle. For example, the innermost pair of fastening indicia may comprise the number “1” printed on the backsheet or the landing zone. Further pairs disposed more outwardly may comprise the next numerals “2” and “3”. Typically three pairs of indicia (1-1, 2-2 and 3-3) are used, however some diapers have a fourth pair of fastening indicia (4-4).

Baby diapers are typically designed for a given weight range of the babies. For example a line-up may start with a first size for new born, size 1 or “NB” (for example up to 5 kg), the next size 2 or “S” being designed e.g. for babies weighing 4 to 8 kg, the next size “M” or Size 3 for babies from 6 to 11 kg, and e.g. the next size “L” or size 4 for babies from 9-14 kg and so on. These values are of course exemplary, as each manufacturer may have a different recommended weight range for the articles in their line-up. There may also be intermediate sizes, for example 4+. As seen in the previous example, the ranges in two neighboring sizes typically overlap at their limits.

The manufacturers may typically design their taped products so that the diaper fits best for babies within the recommended weight range when the tapes are fastened between the innermost indicia and the outermost indicia. Because consecutive weight ranges normally overlap, the typical baby (or design target baby) actually switches sizes at about the midpoint of the overlap. So for example if the S size is <5 kg and the M size is between 4 kg-8 kg, the expected transition weight is about 4.5 kg. In this way the caregiver has the option of switching as early as 4 kg, or as late as 5 kg (depending on her baby's unique needs), but for a typical baby the fastening indicia will be placed such that a 4.5 kg baby would be at the outermost indicia of S size and the innermost indicia of M size. In this way the outermost indicia serve as a reminder that baby should now be well within the design range of the next size up. The intermediate placement of the tapes correspond to the intermediate weight within the range.

While most caregivers understand that the fasteners should be optimally fastened somewhere in the fastening area, caregivers often have difficulty understanding the best placement of the tapes using the fastening indicia. Different proposals have been made in the art to help caregivers making a better choice of the article's size using the indicia. WO 2009/005413A1 (Lenhult et al.) for example addresses the problem of helping the caregivers to determine if the article is of a correct size or whether the wearer needs an article of a larger size. Lenhult uses size fit indicating means locating outwardly of a target area that indicates that the article is too small for the wearer. The size indicating means may comprise an indication as to which size the change should be made. JP 2010148731 (Toda et al.) describes a similar indicia system that helps the caregiver deciding when to change the sizes of the diaper to fit the form of the wearer.

However these systems may be non-intuitive to many consumers and the positioning of the fasteners on the fastening area will still depend on the individual preferences or habits of the caregiver. Therefore there is a need for a simple, easy, intuitively understandable fastening system to provide a good fit with the wearer.

### SUMMARY OF THE INVENTION

The invention is directed to a line-up of personal hygiene absorbent articles comprising at least three packages. The first package comprises articles having a first size, the second package comprises articles having a second size and the third package comprises articles having a third size. Each size has a different weight range, which is the recommended wearer's weight range as indicated by the manufacturer for a given article's size. The weight range of the third size is partially higher than the weight range of the second size, which is partially higher than the weight range of the first size. The upper limit of one weight range is thus higher than or at least equal to the lower limit of the next size's weight range in the line-up. In this way, at least for the sizes claimed and preferably for the whole line-up, two consecutive sizes will have a weight range overlap and thus the line-up provides a continuous weight offering with no gaps in the weight ranges between two neighboring sizes in the line-up.

Each article in the line-up has a front edge, a back edge and is notionally divided by a longitudinal axis extending from the middle of the front edge to the middle of the back edge. The article is notionally divided in a front region and a back region, each region having an equal length as measured along the longitudinal axis. A transversal axis is defined as the axis perpendicular to the longitudinal axis and notionally separating the front region from the back region of the article.

The back region comprises a pair of fasteners that can be fastened to a fastening area on the front region of the article so that the front region and the back region form a waist opening. The distance between the fasteners, herein referred to as wingspan, can be measured along the transversal direction when the diaper is disposed flat as is described further below. The wingspan is measured at rest, i.e. with any elastic elements in the transversal direction un-tensioned and between the distal tips of the fasteners.

The fastening area in the front region comprises an innermost pair of fastening indicia, an outermost pair of fastening indicia and typically at least one intermediate pair of fastening indicia, wherein one member of each pair is disposed on one side of the longitudinal axis and the other member of that pair is disposed symmetrically on the other side of the longitudinal axis. Each member of a given pair is separated from the other member of that pair by a respective distance, referred herein to as spacing.

Each article has an inner waist circumference defined by the sum of the wingspan and the spacing between the innermost fastening indicia, and an outer waist circumference defined by the sum of the wingspan and the spacing between the outermost fastening indicia. According to the present invention, the outer waist circumference of the article of the first size is within 30 mm of the inner waist circumference of the article of the second size, and the outer waist circumference of the article of the second size is within 30 mm to the inner waist circumference of the article of the third size.

The inventors have found that by matching the outer waist circumference of a given size with the inner waist circumference of the next size (within the tolerance given of +/−30 mm or less, e.g. +/−20 mm), the caregivers can more easily attach the fasteners in the right position when changing from one size of diaper to the next. The invention provides for a better attachment of the diaper around the wearer across the line-up.

### BRIEF DESCRIPTION OF THE DRAWINGS

FIG. 1 shows a perspective view of an exemplary taped diaper in a closed position as it would be when worn by a wearer;

FIG. 2 shows a flat view of the garment-facing side of the exemplary taped diaper of FIG. 1;

FIGS. 3, 4 and 5 show close-up views of exemplary fastening areas with the fastening indicia for a first, a second and a third size respectively in increasing size order; these drawings are to scale relative to each other;

FIG. 6 schematically shows an example of a line-up of three packages comprising absorbent articles of different sizes.

### DETAILED DESCRIPTION OF THE INVENTION

### General Description of an Absorbent Article

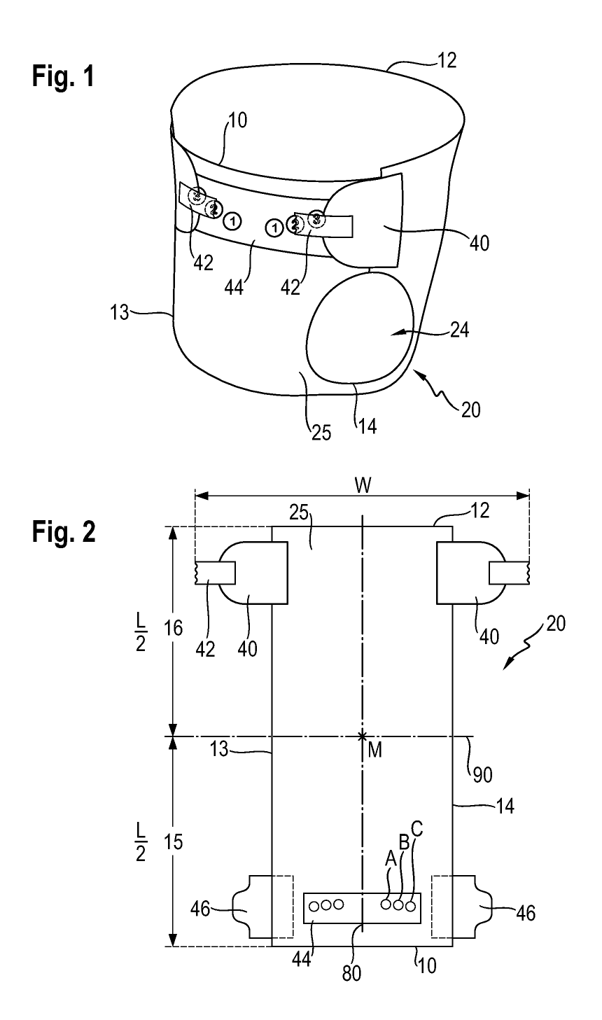

An exemplary absorbent article according to the invention in the form of a baby taped diaper 20 is represented in FIGS. 1-2. FIG. 1 is a perspective view of the exemplary diaper in a closed state as it would appear when worn by a wearer. FIG. 2 shows the same diaper flat in an open configuration with the garment-facing side of the diaper (the backsheet) represented. This diaper 20 is shown for illustration purpose only, as the invention may be used in a wide variety of diapers or other absorbent articles such as adult incontinence diapers. In the following, the word “diaper” and “absorbent article” are used interchangeably.

The absorbent article 20 comprises a front edge 10, a back edge 12, and two longitudinally-extending side (lateral) edges 13, 14. The front edge 10 is the edge of the article which is intended to be placed towards the front of the user when worn, and the back edge 12 is the opposite edge. The absorbent article is notionally divided by a longitudinal axis 80 extending along a longitudinal direction from the middle of the front edge to the middle of the back edge of the article and dividing the article in two substantially symmetrical right half and left half relative to this axis, when viewing the article in a flat out configuration, as exemplarily shown in FIG. 2.

The article has a length L as measured along the longitudinal axis 80 from the back edge to the front edge as shown on FIG. 2. If some parts of the article are under tension due to elasticized components, the article may be typically flattened using clamps along the periphery of the article and/or a sticky surface, so that the article can be pulled taut so as to be substantially flat. The absorbent article can also be notionally divided by a transversal axis 90 into a front (waist) region 15 extending from the transversal axis 90 to the front edge 10 of the article and a back (waist) region 16 extending from the transversal axis 90 to the back edge 12 of the article. The transversal axis is 90 defined as the line perpendicular to the longitudinal axis 80 and placed at half the length of the article so that the front region and the back region have the same length. The intersection of the longitudinal axis 80 and the transversal axis 90 is defined herein as the middle point M of the article. The fasteners 42 are separated by a distance W, referred to as wingspan in the following, measured along the transversal direction at their furthest away points (tip to tip) as illustrated on FIG. 2. If the article has an elasticized back waistband or back ears, the wingspan is measured with the waistband and back ears at rest, in other words in a non-stretched state, with the back edge of the article oriented as straight as possible parallel to the transversal direction.

The absorbent articles of the invention may comprises any of the typical layers and components used in absorbent products of the diaper type, and which are not necessarily represented in the simplified FIGS. 1-2. Typically, absorbent articles will comprise on their wearer-facing surface a liquid permeable topsheet 24, on their garment-facing surface a liquid impermeable backsheet 25 and an absorbent core 28 between the topsheet and the backsheet, referred together as the chassis of the diaper. The backsheet may typically comprise a fluid impermeable plastic film, which may be printed with a backsheet pattern, and a low basis weight (8-25 gsm) nonwoven cover to give a nicer feel and appearance to the backsheet. The absorbent article may also comprise a fluid acquisition layer and/ or a fluid distribution layer between the topsheet and the absorbent core. Other typical diaper components are not represented such as elasticized gasketing cuffs, upstanding barrier leg cuffs but are typically present. The absorbent article may also comprise other usual components, which are not represented in the Figures, but may be present if it is desired to increase the performance of the article, transverse barrier cuffs, a lotion application, channels in the core and/or the distribution layer, a wetness indicator, etc., all these components are well known in the art.

The absorbent article may also comprise an elastic waist band (also called elastic waist feature) disposed parallel to and along the back edge of the article and less commonly another elastic waist band parallel to and along the front edge of the article. Such waistbands help providing improved fit and containment at the back and/or front edge of the article. The elastic waist feature is generally intended to elastically expand and contract to dynamically fit the wearer's waist. The elastic waist feature may be constructed in a number of different configurations. Non-limiting examples of back and front waistbands can be found in WO 2012/177400 and WO 2012/177401 (Lawson), and U.S. Pat. No. 4,515,595, U.S. Pat. No. 4,710,189, U.S. Pat. No. 5,221,274 and U.S. Pat. No. 6,336,922 (VanGompel et al.).

The topsheet 24, the backsheet 25, the absorbent core 28 and the other article components may be assembled in a variety of well-known configurations, in particular by gluing, fusion and/or pressure bonding.

### General Description of a Fastening System 42, 44

The absorbent article includes a fastening system that allows the article to be closed around the body of the wearer, thus providing openings for the legs and the waist of the wearer as shown on FIG. 1. The fastening system also provides lateral tensions about the circumference of the absorbent article to hold tightly the absorbent article on the wearer. The fastening system comprises a pair of fasteners 42 disposed on or attached to the back region of the article that can be fastened to a fastening area 44 on the garment-facing side of the front waist region of the article. The fasteners may for example comprise a pair of conventional fastening tapes 42, comprising at their distal end hook elements that can interact with loop elements of a hook-and-loop system. The fastening area 44, also called landing zone, is provided on the garment-facing side of the front region of the article. The fasteners 42 may be directly or indirectly attached to the chassis of the article. In particular, fastening tapes may be attached to the chassis of the article through back ears 40 that may be typically stretchable.

The fasteners 42 can typically be releasably attached to the fastening area 44. The fastening area may in particular comprise loop components adapted to engage with hook components of the fasteners in a hook-and-loop fastening system, as is commonly used in the field. More generally any fastener systems comprising a male component and a female component may be used, such as tabs & slots, buckles, buttons, snaps, and/or hermaphroditic fastening components. Other known fastening systems may also be used, for example fasteners comprising a contact glue with low adhesion force (such as are used on sticky notes) with the fastening area optionally comprising a co-adhesive. Fasteners comprising micro-suction cups are also known.

The absorbent article may comprise front ears 46 and/or back ears 40, which are disposed close to the front and back edge of the diaper respectively and extend laterally externally from the rest of the chassis. The front ears and /or back ears can be formed integrally from the chassis, for example formed from the topsheet and/or backsheet as side panels. This is typically the case when the chassis is not rectangular, but sand-clock or similarly shaped. In that case, the fastening tapes may be instead directly attached to the chassis instead of indirectly via the back ears. Alternatively, as represented in FIGS. 1-2, the front and/or back ears may be separate elements attached by gluing and/or heat embossing to the rest of the article.

The back ears 40 are optionally stretchable to facilitate the attachment of the tabs 42 on the landing zone 44 and maintain the taped diapers in place around the wearer's waist. The back ears 40 may be stretchable and elastic. The back ears may comprise for example a laminate comprising an elastomeric film, typically sandwiched between two nonwoven layers. The laminate may be mechanically activated for example by ring rolling. Exemplary disclosures of such elastic laminates can be found in WO 2007/122588 (Mansfield) or WO 2006/124337 (Venkitaraman et al.). Back ears are typically used when the chassis of the diaper is rectangular as illustrated in FIG. 2 and are disposed between the fastening tapes and the chassis of the article. The front ears are typically not elastic or extensible and may be made of a nonwoven material. More rarely, some products are proposed having a double fastening system, for example having an additional hook patch on the front ears which engages with the back ears nonwoven. The fasteners required by the invention are those disposed at the outboard-most position on the back region of the article. It has also been suggested in alternative products, mostly adult incontinence products, to have secondary fastening tapes located longitudinally inboard of the primary fastening tapes, these having a corresponding landing zone. Any of these fastening tapes and corresponding landing zones may be according to the invention.

The fastening area in the front region may be constituted by a dedicated piece of material 44 adhesively or otherwise attached to the external facing side of the backsheet, referred herein as the landing zone. This landing zone material may be generally rectangular with its long side disposed transversally relative to the longitudinal axis. This landing zone material typically comprises fibers that form the female component of the fastening system that can releasably engage with the male component on the fastening tabs. Various landing zone materials have been developed in the past that can be used in the present invention. The landing zones may for example comprise a plastic film onto which a knitted fiber mesh has been attached for example by an adhesive. The fastening indicia may be printed on the plastic film. A nonwoven may also be used alone or in combination with a plastic film to form a laminate. Suitable nonwoven fabrics comprising crimped conjugate fibers are for example disclosed in US 2004/0067709A1 (Kishine et al.).

The landing zone material may be semi-transparent or opaque. The fastening indicia may be printed or otherwise formed on the landing zone material itself, or if the landing zone material is at least partially transparent, the fastening guide may be printed on the backsheet film as part of a backsheet pattern and may be visible through the dedicated fastening area material. US 2010/298,796A1 (Horn et al.) for example discloses a graphic image that can be printed directly on a nonwoven landing zone material. Backsheet typically comprises a decorative pattern printed on the film material, especially in children diapers, which is not further discussed herein.

Alternatively, it is also possible that the fastening area is not formed by a dedicated separate piece of material forming a landing zone, but that instead the fasteners may directly engage with the fibers of the nonwoven outer cover of the backsheet, if such an outer cover is present. This is for example disclosed in WO 2016/060922A1 (Jennewein et al.). In that case the fastening indeed may be printed on the backsheet film as part of the backsheet printed pattern, and the fastening area is not limited to a particular area of the front region. In summary, any type of fastening systems known in the art for diapers may be used.

### Line-Up

The term “line-up” as used herein refers to a plurality of packages comprising absorbent articles with increasing size ranges and having like packaging elements, which may in particular be brand name (e.g. Pampers®) and if applicable sub-brand (e.g. Pamper® Baby-Dry®) but also including packaging material type, film, paper, dominant color, design theme, etc. The common packaging elements convey to the users that the different products are associated to each other, so that when an article of a certain size becomes too small for a given wearer, the user will directly understand that the article of the next size in the line-up is adapted to a larger wearer.

The size of the absorbent articles may be typically reported by numerals, “size 0” (or “new baby”), “size 1”, “size 2” . . . , or letters “XS”, “NB” (New Baby), “S”, “M”, “L”, . . . or otherwise. Typically, each size will have a targeted or recommended wearer weight range to which they are particularly adapted. This recommended weight range is indicated by the manufacturer on the packaging of the absorbent articles. The weight ranges of two neighboring sizes overlap partially, as will be illustrated in the example further below. The targeted weight range indicated will be determined by the manufacturer, which will adapt the dimensions and types materials for the need of the wearer in the targeted ranges. For example, the diaper's dimensions and the amount of absorbent material will generally increase for baby diapers of increasing sizes, at least until the toilet training stage at which point the amount of absorbent material may be reduced as the child learns to control its incontinence.

The articles of the first, second and third sizes of the invention may advantageously directly follow one another in the line-up's progression, for example the first size may be “S”, the second size may be “M” and third size may be “L”. The recommended weight range of the first size overlaps at its higher limit with the weight range of the second size, and the weight range of the second size overlaps at its higher limit with the weight range of the third size. In other words, the higher limit of the range of the first size may be higher than or equal to the lower limit of the range of the second size, and the higher limit of the range of the second size may be higher than or equal to the lower limit of the range of the third size. For example, the recommended weight range for a first article (“NB” =New Baby) may be up to 5 kg, the weight range for the next size (“S”=small size) may be from for 4-8 kg, thus having an overlap in the 4-5 kg range. The invention example below gives further example across size S, M and L. Intermediate size(s) may also be present in the line-up, for example size 4+. These intermediate sizes may or may not be according to the invention.

### Fastening Indicia

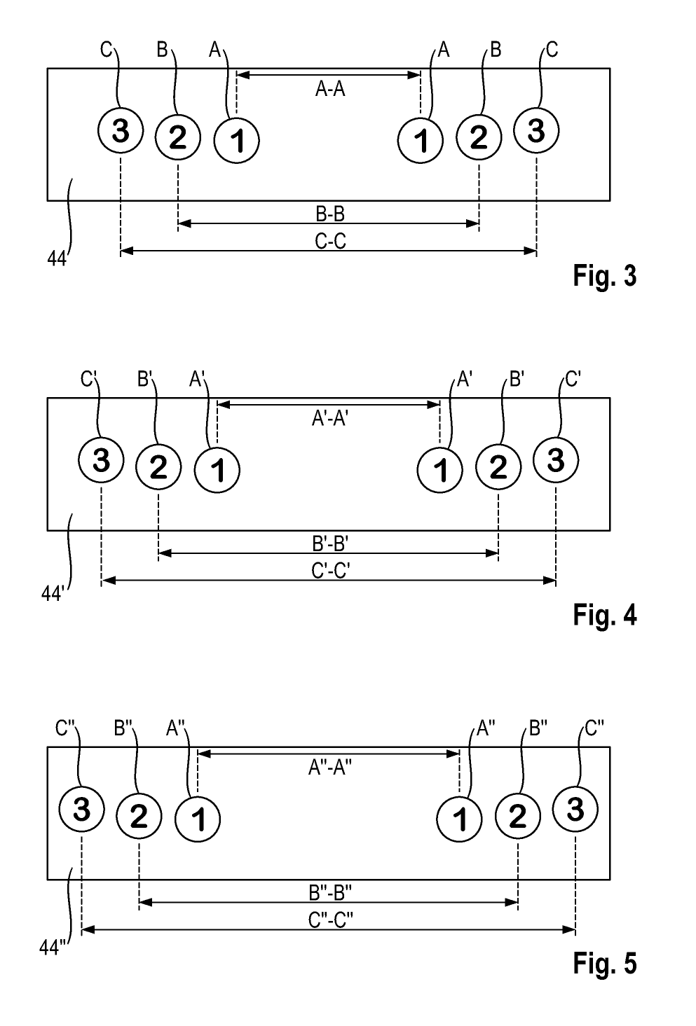

Fastening indicia are distinctive marks that provide an indication to the user of possible attachment positions of the fasteners on the fastening area. The term “user” as used herein refers to the person putting the article on the wearer, which is typically a caregiver such as a parent, educational staff or a nurse. The fastening indicia may typically comprise a visual symbol, signal or cue that is able to indicate a progression. Such indicia may take many possible different forms. As shown in the examples as represented in FIGS. 3-5, the fastening indicia may simply comprise written Arabic numbers “1”, “2” and “3”, but any other symbols immediately understandable for the depiction of a progression are also suitable such as roman numbers (“I”, “II”, “III”), alphabetical letters (“A”, “B”, “C”), any objects or items in varying numbers (one heart, two hearts, etc.), increasingly larger animals (a rabbit, a larger dog, an elephant), variations of shading (light, medium, strong) or color coding (green, yellow, red) to name a few. While not always necessary, the fastening indicia may also comprise a geometric shape having a clearly identifiable center such as a circle or a regular polygon (square, rectangle, hexagon, etc.). The symbol that indicates the progression may then be disposed centered relative to the geometrical shape.

Alternatively, the fastening indicia do not need to inherently provide an indication of a progression because it is typically immediately apparent for the users that placing the fasteners towards the center of the fastening area will result in a smaller waist opening relative to a more outward placement of the fasteners. Thus the plurality of fastening indicia may simply indicate possible positioning of the fasteners without connoting a progression. For example the fastening indicia may be a graphic connoting attachment such as a button, a pin or zipper or any decorative graphic such as a flower, an animal or a toy. The fastening indicia may also be comprised of different zones without a particular symbol, for example zones of different colors. The distances between the indicia in this case is measured at the transversal center of each zone, in a consistent manner for each zone and each size. The fastening indicia also help placing the fasteners symmetrically on each side of the article by providing elements of reference for the user.

FIG. 3 shows an exemplary landing zone comprising three pairs of indicia A-A, B-B, C-C. In this example, the indicia are printed and comprise the numbers “1”, “2” and “3” respectively, and are disposed in circle which are visible on the fastening area. The indicia are present as pairs of indicia, with one member of each pair disposed on one side of the longitudinal axis, and the other member disposed on the other side of the longitudinal axis. Each pair of fastening indicia is thus symmetrically disposed relative to the longitudinal axis. The indicia may be contained in a circle or another regular geometric shape in order to highlight their position and the center of the indicia. The fastening area may of course also comprise a decorative pattern in addition to the fastening indicia. The decorative pattern may also comprise elements that do not cooperate with the indicia, for example the brand name or logo of the manufacturer may be displayed between the indicia in the center of the fastening area.

The innermost pair of fastening indicia is the pair “1-1” separated by a distance A-A, the outermost pair of fastening indicia is the pair “3-3” separated by the distance C-C. In this example, there is one intermediate pair of fastening indicia “2-2” between the innermost and outermost indicia, referred to in the Figures by a separated distance B-B. The distance between two indicia is measured perpendicularly to the longitudinal direction of the diaper, in other words is parallel to the transversal direction. The plurality of the individual indicia A, B, C on each side of the fastening area may be aligned along a notional line. This line may be aligned parallel to the transversal direction of the article, or at an angle relative to the transversal axis, as illustrated on FIG. 3, such an angle ranging for example from 1° to 20°.

### Inner and Outer Waist Circumferences

According to the present invention, it was found that the circumference of the waist opening should remain about the same at the time of changing from one size to the next up, within the given tolerance as indicated in the claim. In this way, the user of the diapers having reached the outermost indicia of the smaller size will be provided with a waist opening of about the same circumference when moving to the next size and attaching the fasteners on the innermost indicia of this bigger size. The inventors have found that the caregiver is thus more likely to place the fasteners at the right position on the bigger size as he or she will recognize that the circumference remain about equal with what he or she experienced last on the smaller size. According to the present invention, “about the same” means that the dimensions of the circumferences are within 30 mm of each other (plus or minus), in particular within 25 mm, or 20 mm, or 15 mm, or even 10 mm of each other. Exemplary values are provided in the example section below of line-up according to the invention and of the prior art.

The inner and outer waist circumferences are calculated by adding the value of the wingspan W to the spacing between the innermost indicia and the spacing between the outermost indicia, respectively, for an article of a given size. As indicated previously, the wingspan is defined by the distance between the extremities of the fasteners along the transversal direction, as illustrated on FIG. 2. The measurement of the wingspan is made with the article laid flat on a table and the back edge of the article substantially aligned with the transversal direction. The back region of the article laid as flat as possible and the fasteners lined up in a straight line, with any components stretchable in the transversal direction (e.g. elastic waistband and consolidated back ears) left in the un-tensioned resting state. While the exact conditions for the measurements of the wingspans are not critical, they should be repeated across the different sizes so that any bias in the measurement method is canceled when comparing the different circumferences values. The same applies for the measurement of the spacing between the indicia.

### Indicia—Landing Zone

The fastening indicia comprises at least two pairs of indicia (innermost and outermost) disposed symmetrically relative to the longitudinal axis of the diaper. Typically there will also be an intermediate pair of fastening indicia B, B in addition to the innermost pair of fastening indicia A, A and the outermost pair of fastening indicia C, C. There may also be for example a second pair of intermediate indicia (not represented). Caregivers intuitively believe that the innermost indicia are the right starting position to place the fasteners for a given size. For example, when a parent starts using a new size of diaper (e.g. “S” size), he or she will place the fasteners on or close to the innermost indicia as the perceived optimum placement. Therefore the innermost indicia should be positioned on the fastening area so that when the center of the fasteners (e.g. hook portion of the tapes) are positioned on the innermost indicia, the position of the fasteners is adapted for the typical entry weight of the targeted weight range of the product at that given size.

As the baby grows, increasing its waist size, the caregiver will progressively shift outwardly the placement of the fasteners on the fastening area to maintain about the same waist tension of the diaper as in the starting position. Once the fasteners reach the outermost indicia, this acts as a cue to the caregivers that the diapers may no longer be adapted to the size of the baby and that it is time to switch to the next size (for example from “S” to “M”). As the caregiver uses the next size diaper, he or she will typically put the fasteners on the innermost indicia on the fastening area of this next size. Therefore the outermost indicia should be positioned on the fastening area so that when the fasteners are positioned centered on the outermost indicia, the position of the fasteners are adapted for the typical exit weight of the targeted weight range of the product at that given size.

The present inventors have also found that it was advantageous that the spacing A-A between the innermost indicia A, A should also increase with increasing product sizes. In this way, when placing the fasteners on the new indicia A′, A′ of the larger size article, the spacing A′-A′ between the new indicia A′, A′ is larger than the spacing A-A on the previous diaper's size (A′-A′>A-A). By moving up again to the next size, the spacing A″-A″ between the innermost indicia A″, A″ of the next size will again be larger than the spacing A′-A′ of the previous size (A″-A″>A′-A′). This is exemplary illustrated in FIG. 3-5, which shows the indicia and fastening area in the form of landing zone for three different sizes of diapers (“S”, “M” and “L”). The three Figures are to scale relative to each other, and illustrate that the innermost indicia's spacing increases with the article's size. As represented, the innermost indicia's spacing A-A shown on FIG. 3 for the first size is smaller than the innermost indicia's spacing A′-A′ for the second size as shown on FIG. 4, and the innermost indicia's spacing increases again for the larger spacing A″-A″ for the third size compared to the first and second sizes. This may be summarized by the equation A″-A″>A′-A′>A-A. The inventors have found that this regular progression of the innermost indicia spacing across the (at least three) increasing sizes of the diaper line-up resulted in a better placement of the fastening tapes relative to the landing zone by the caregivers.

Advantageously, the spacing C-C between the members of the outermost pair of fastening indicia will also increase with the size similarly to the spacing between the members of the innermost pair, and likewise for the spacing between the one or more intermediate pair(s) of indicia B-B. Furthermore, for each article of a given size, the distances between two neighboring indicia on the same side of the longitudinal axis may be substantially the same for all neighboring indicia (A-B=B-C). The neighboring distance between two neighboring indicia may also be the same for the first, second, and third size (A-B=A′-B′=A″-B″=B-C=B′-C′=B″-C″).

The spacing between two indicia is measured between the centers of each indicia, also known as “target”, when the indicia have a clearly identifiable center. For example, if the indicia comprises a regular geometric figure such as a circle as represented in the Figures or a square or a polygon, the distance is measured between each center of this geometric figure. If the indicia consists only of a numerical sign, the distance is measured in the middle of numerical sign in the transversal direction. While it is not possible to indicate a measurement principle for all imaginable other type of indicia having irregular shape, this is also not critical as what is measured for the invention is in an increase or decrease of the spacing or circumferences across sizes, rather than an absolute value. Thus the spacing between two indicia should be measured consistently using the same reference points for the different indicia.

While the indicia may be aligned parallel to the transversal direction or at an angle to the transversal direction or otherwise, the spacing between two indicia is measured as reported parallel to the transversal direction, as illustrated in FIGS. 3-5. In order to account for measurement uncertainty, two indicia spacing are considered to be substantially the same is they differ by 2 mm or less. Two indicia spacing are considered to be different if they differ by more than, in particular 3 mm or more, or 4 mm or more.

As further consideration, when conventional fastening tapes with a hook portion are used as fasteners, the two innermost indicia can advantageously be disposed on the fastening area so that the caregiver can place the center of the hook portions on the center of the innermost indicia without overlapping fastening tapes. The desired end-tape-to-end-tape spacing may be set at a minimum of 10 mm when the hook portion of the tapes is disposed on the center of the innermost indicia. This avoids an overlap of the tape ends. Furthermore, an exemplary value for the distance center-of-hook portion to end of the tape may be about 15 mm. With the desired gap of at least 10 mm between the tapes ends indicated above, the minimum center-to-center spacing of the innermost indicia may thus for example be at least 40 mm, alternatively at least 50 mm.

In the illustrations of FIGS. 3-5, the fastening area comprises a landing zone material 44, 44′, 44″. The landing zone material and its dimension may be the same across the sizes, as represented, but may be also different. The indicia may be printed on the landing zone material 44 or on the backsheet underneath if the landing zone material is sufficiently transparent to keep the indicia visible through the landing zone. However it is also possible that there is no landing zone 44 if the fasteners can engage directly with the nonwoven backsheet outer cover which then directly form a fastening area. In that case the indicia may be printed directly on the backsheet film or backsheet nonwoven under the landing zone material. The landing zone/fastening area may be larger in the transversal direction than the region where the fastening indicia are disposed, to allow fastening laterally inwards of the innermost fastening indicia and laterally outwards of the outermost fastening indicia, if desired by the caregiver.

### INVENTION EXAMPLE 1

The following is an example of a line-up of taped diapers comprising four sizes (NB=“New Baby”, “S”=Small, “M”=Medium and “L”=Large). The fastening area on all products comprise a landing zone (LZ) material and three pairs (innermost 1-1, outermost 3-3 and intermediate 2-2) of indicia. The articles may be generally as illustrated in the Figures. Note that while this line-up comprises four sizes that fulfill the requirement of the invention, the present invention merely requires that at least three sizes fall under the requirement of the claims. All distances in all the examples are reported in mm.

| | Size and recommended weight range | | | |
|---|---|---|---|---|
| | NB (up | S | M | L |
| | to 5 kg) | (4-8 kg) | (6-11 kg) | (9-14 kg) |
| Wingspan | 305 | 336 | 356 | 376 |
| Innermost indicia spacing | 40 | 55 | 75 | 75 |
| A-A | | | | |
| Intermediate indicia spacing | 63 | 80 | 90 | 110 |
| B-B | | | | |
| Outermost indicia spacing | 87 | 100 | 110 | 124 |
| C-C | | | | |
| Inner waist circumference | 345 | 391 | 431 | 461 |
| Outer waist circumference | 392 | 436 | 466 | 500 |
| Difference between the | — | 1 | 5 | 5 |
| Outer waist circumference | | | | |
| of a given size and the Inner | | | | |
| waist circumferences of the | | | | |
| next size | | | | |

Inner waist circumference=Wingspan+Innermost indicia spacing A-A

Outer waist circumference =Wingspan +Outermost indicia spacing C-C

The difference between the Outer and Inner Waist Circumference is reported in absolute value so that it is irrelevant which value is subtracted to the other. According to the invention claimed, the difference should be less than 30 mm for at least three consecutive articles in the line-up, but may be advantageously even lower, as exemplified in this first example.

According to preferred but not limiting aspect of the invention, the spacing between the members of each pairs (innermost, intermediate and outermost) increases progressively as the product's size increase.

### PRIOR ART EXAMPLE 1

The following measurements were made on a line-up of Moony® diapers manufactured by the Unicharm Company and sold in Japan. The fastening area consisted of a split landing zone (two similar material separated by a gap).

| | Size and recommended weight range | | | |
|---|---|---|---|---|
| | NB (up | S | M | L |
| | to 5 kg) | (4-8 kg) | (6-11 kg) | (9-14 kg) |
| Wingspan | 249 | 276 | 284 | 304 |
| Innermost indicia spacing | 56 | 54 | 65 | 65 |
| A-A | | | | |
| Intermediate indicia spacing | 96 | 94 | 106 | 105 |
| B-B | | | | |
| Outermost indicia spacing | 137 | 135 | 147 | 146 |
| C-C | | | | |
| Inner waist circumference | 305 | 330 | 349 | 369 |
| Outer waist circumference | 386 | 411 | 431 | 450 |
| Difference between the | — | 56 | 62 | 62 |
| Outer waist circumference | | | | |
| of a given size and the Inner | | | | |
| waist circumferences of the | | | | |
| next size | | | | |

### PRIOR AFT EXAMPLE 2

The following measurement were made on Merries® diapers marketed in China by the Kao Corporation.

| | Size and recommended weight range | | | |
|---|---|---|---|---|
| | NB (up | S | M | L |
| | to 5 kg) | (4-8 kg) | (6-11 kg) | (9-14 kg) |
| Wingspan | 301 | 321 | 344 | 354 |
| Innermost indicia spacing | 41 | 41 | 56 | 55 |
| A-A | | | | |
| Intermediate indicia spacing | 86 | 84 | 97 | 96 |
| B-B | | | | |
| Outermost indicia spacing | 130 | 126 | 137 | 136 |
| C-C | | | | |
| Inner waist circumference | 342 | 362 | 400 | 409 |
| Outer waist circumference | 431 | 447 | 481 | 490 |
| Difference between the | — | 69 | 47 | 72 |
| Outer waist circumference | | | | |
| of a given size and the Inner | | | | |
| waist circumferences of the | | | | |
| next size | | | | |

### PRIOR ART EXAMPLE 3

The following measurements were made on Pampers® diapers marketed in Japan by the Procter & Gamble Company before the filing date of this patent application.

| | Size and recommended weight range | | | |
|---|---|---|---|---|
| | NB (up | S | M | L |
| | to 5 kg) | (4-8 kg) | (6-11 kg) | (9-14 kg) |
| Wingspan | 311 | 352 | 346 | 368 |
| Innermost indicia spacing | 41 | 51 | 51 | 51 |
| A-A | | | | |
| Intermediate indicia spacing | 71 | 88 | 87 | 88 |
| B-B | | | | |
| Outermost indicia spacing | 101 | 126 | 12 | 125 |
| C-C | | | | |
| Inner waist circumference | 352 | 403 | 397 | 419 |
| Outer waist circumference | 412 | 478 | 470 | 493 |
| Difference between the | — | 9 | 81 | 52 |
| Outer waist circumference | | | | |
| of a given size and the Inner | | | | |
| waist circumferences of the | | | | |
| next size | | | | |

### Packages

Absorbent articles for personal hygiene are typically packaged by the manufacturer in a plastic bag and/or a cardboard box for transport and sale. The articles may also be folded before being packaged to save space as is known in the art. The back and front ears of taped diapers are for example typically folded inwardly before bi-folding the diaper along its transversal axis before being packaged. The absorbent articles may be packaged under compression, so as to reduce the size of the package so that the caregivers can easily handle and store the packages, while also providing distribution and inventory savings to manufacturers owing to the size of the packages. The first, second and third package may for example comprise each from 2 to 200 of the first, second and third articles respectively.

Most typically, the absorbent articles of the invention will be marketed to the consumers as a display of packages comprising the disposable articles of different sizes and having like article constructions such as the same brand and/or sub-brand and/or product features and/or benefits across the line-up, as indicated previously. The packages will be typically sold in proximity to each other in a given area of a retail store, or if sold in a virtual internet store, the packages of different sizes may be displayed or linked to from the same internet page.

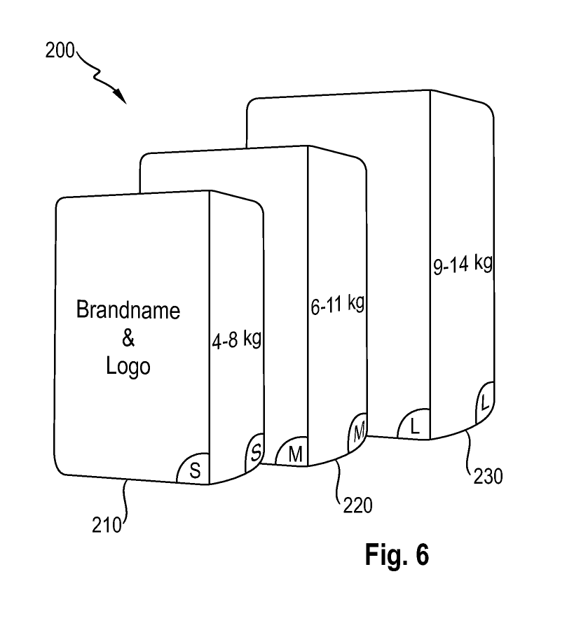

FIG. 6 shows a schematic view of a line-up 200 comprising three packages 210, 220, 230 each comprising three different sizes S, M and L of absorbent articles respectively. Of course the whole line-up may typically comprises more than sizes, for example additionally a smaller size (NB) and larger sizes (XL, XXL), which are not represented. Of course, each manufacturers may use different size designation, for example it is more common to use numbering such as size 0, 1, 2, 3 for a line-up in Europe and Americas. The dimensions of the packages may be in relation to the size of the absorbent articles packaged therein, but this is not necessary always the case as the quantity of articles may vary in each package.

### Miscellaneous

As used herein, the terms “comprise(s)” and “comprising” are open-ended; each specifies the presence of the feature that follows, e.g. a component, but does not preclude the presence of other features, e.g. elements, steps, components known in the art or disclosed herein. These terms based on the verb “comprise” should be read as encompassing the narrower terms “consisting essentially of” which excludes any element, step or ingredient not mentioned which materially affect the way the feature performs its function, and the term “consisting of” which excludes any element, step, or ingredient not specified. Any preferred or exemplary embodiments described below are not limiting the scope of the claims, unless specifically indicated to do so. The words “typically”, “normally”, “preferably”, “advantageously”, “in particular” and the likes also qualify features which are not intended to limit the scope of the claims unless specifically indicated to do so.

Unless indicated otherwise, the description and claims refer to the absorbent core and article before use (i.e. dry, and not loaded with a fluid) and conditioned at least 24 hours at 21° C.+/−2° C. and 50+/−5% Relative Humidity (RH).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A line-up of personal hygiene absorbent articles, wherein the line-up comprises at least three packages, the first package comprising articles having a first size, the second package comprising articles having a second size and the third package comprising articles having a third size, each size having a different weight range, wherein the weight range of the third size is at least partially higher than the weight range of the second size, and the weight range of the second size is at least partially higher than the weight range of the first size;
wherein each article in the line-up has a front edge, a back edge and is notionally divided by a longitudinal axis extending from the middle of the front edge to the middle of the back edge, and the article can be notionally divided in a front region and a back region, each region having an equal length as measured along the longitudinal axis,
wherein the back region comprises a pair of fasteners that can be fastened to a fastening area on the front region of the article so that the front edge and the back edge form a waist opening;
wherein the distance between the pair of fasteners at rest defines a wingspan in the transversal direction perpendicular to the longitudinal direction;
wherein the fastening area in the front region comprises an innermost pair of fastening indicia and an outermost pair of fastening indicia, wherein one member of each pair is disposed on one side of the longitudinal axis and the other member of that pair is disposed symmetrically on the other side of the longitudinal axis; wherein the two members of each pair are separated by a respective distance;
wherein each article has an inner waist circumference defined by the sum of the wingspan and the spacing between the innermost fastening indicia and an outer waist circumference defined by the sum of the wingspan and the spacing between the outermost fastening indicia;
wherein the outer waist circumference of the article of the first size is within 30 mm of the inner waist circumference of the article of the second size, and the outer waist circumference of the article of the second size is within 30 mm of the inner waist circumference of the article of the third size.

2. A line-up according to claim 1, wherein the outer waist circumference of the article of the first size is within 20 mm of the inner waist circumference of the article of the second size, and the outer waist circumference of the article of the second size is within 20 mm to the inner waist circumference of the article of the third size.

3. A line-up according to claim 2, wherein the outer waist circumference of the article of the first size is within 10 mm of the inner waist circumference of the article of the second size, and the outer waist circumference of the article of the second size is within 10 mm to the inner waist circumference of the article of the third size.

4. A line-up according to claim 1, comprising a fourth package comprising articles having a fourth size, wherein the weight range of the fourth size is at least partially higher than the weight range of the third size, and the outer waist circumference of the article of the third size is within 30 mm of the inner waist circumference of the article of the fourth size.

5. A line-up according to claim 4, wherein the outer waist circumference of the article of the third size is within 20 mm of the inner waist circumference of the article of the fourth size.

6. A line-up according to claim 5, wherein the outer waist circumference of the article of the third size is within 10 mm of the inner waist circumference of the article of the fourth size.

7. A line-up according to claim 1, wherein the spacing between the innermost pair of fastening indicia of the articles of the third size is greater than the spacing between the innermost pair of fastening indicia of the articles of the second size, and the spacing between the innermost pair of fastening indicia of the articles of the second size is greater than the spacing between the innermost pair of fastening indicia of the articles of the first size.

8. A line-up of absorbent articles according to claim 1, wherein the spacing between the outermost pair of fastening indicia of the articles of the third size is greater than the spacing between the outermost pair of fastening indicia of the articles of the second size, and the spacing between the outermost pair of fastening indicia of the articles of the second size is greater than the spacing between the outermost pair of fastening indicia of the articles of the first size.

9. A line-up of absorbent articles according to claim 8, wherein the fastening area of each article further comprises one or more pair of intermediate indicia between the innermost pair of fastening indicia and the outermost pair of fastening indicia.

10. A line-up of absorbent articles according to claim 9, wherein the three neighboring indicia on the same side of the longitudinal axis are linearly aligned.

11. A line-up of absorbent articles according to claim 1, wherein the fastening indicia comprise a numerical indicia, and the numerical indicia increases from the innermost indicia to the outermost indicia.

12. A line-up of absorbent articles according to claim 1, wherein the fastening indicia comprise a regular geometrical shape having a center such as a circle or a regular polygon.

13. A line-up of absorbent articles according to claim 1, wherein for each article the distances between two neighboring indicia on the same side of the longitudinal axis is substantially the same for all neighboring indicia.

14. A line-up of absorbent articles according to claim 1, wherein the first size article, the second size article and the third size article each comprises exactly a pair of innermost indicia, a pair of outermost indicia, and a pair of intermediate indicia.

15. A line-up of absorbent articles according to claim 1, wherein each article comprises a topsheet, a backsheet and an absorbent core between the topsheet and the backsheet, and wherein the fastening area comprises a landing zone material attached to the external side of the backsheet.

16. A line-up of absorbent articles according to claim 15, wherein the fastening indicia are printed on the landing zone material.

17. A line-up of absorbent articles according to claim 15, wherein the fastening indicia are printed on the backsheet and are visible through the landing zone material.

18. A line-up according to claim 1, wherein the first, second and third packages each comprises from 2 to 200 of the first, second and third articles respectively.
